(19) United States
(12) Patent Application Publication
(10) Pub. No.: US 2016/0129238 A1
(43) Pub. Date: May 12, 2016

(54) METHOD TO OPTIMIZE ELECTRODE PLACEMENT FOR CRANIAL ELECTRICAL STMULATION

(71) Applicant: Joel Steven Goldberg, Hillsborough, NC (US)

(72) Inventor: Joel Steven Goldberg, Hillsborough, NC (US)

(21) Appl. No.: 14/995,376

(22) Filed: Jan. 14, 2016

(51) Int. Cl. A61N 1/04

(52) U.S. Cl. CPC A61N 1/0456

(57) ABSTRACT

A method for optimum preparation and placement of transcranial electrodes is described. The method consists of application of liquid silver paint to standard monitoring electrodes and placement of bilateral electrodes over the pterions, the thinnest areas of the skull. This method minimizes the impedance between electrodes facilitating low power stimulation of the brain as measured by the phosphene threshold that has been redefined in this invention. The locations of the pterions vary between racial groups, but in most cases the pterions overlie the anterior temporal lobes. The anterior temporal lobe is contiguous with the amydala and hippocampus and stimulation of these limbic system structures has been known to produce fear, along with other altered states of consciousness. Low power square wave stimulation via these electrodes produces phosphenes at frequencies closely approximating the Schumann resonance frequency and its harmonics and theta, alpha, beta and gamma EEG frequencies of the brain. External electrical stimulation of the brain from changing electromagnetic fields may be targeted to the amydala or hippocampus and produce fear that is crucial for survival.

### CROSS-REFERENCES TO RELATED APPLICATIONS

None

### FEDERALLY FUNDED RESEARCH

Not applicable

### BACKGROUND OF THE INVENTION

Transcranial electrical stimulation is a common modality used for the treatment of depression, anxiety, insomnia and chronic pain. Problems with this therapy include standardization of cutaneous electrode placement and optimization of the stimulation parameters of waveform, frequency, current and voltage. Present methods to position transcranial electrodes use a standard EEG montage and target the primary motor cortex for treatment of chronic pain and the dorsolateral prefrontal cortex for the treatment of depression. (DaSilva, Volz, Bikson, & Fregni, 2011)

Controversy exists whether a targeted stimulus to the brain is more efficacious than global brain stimulation. In transcutaneous magnetic stimulation (TMS) global or targeted brain stimulation produces similar clinical outcomes. Electroconvulsive therapy produces global stimulation with a seizure response that is efficacious for the treatment of depression.

There are theoretical reasons to minimize the impedance between the stimulating electrodes. Decreasing the impedance between electrodes has the advantage of producing stimulation with low electrical power and may be targeted to underlying structures. Lower electrical power used for stimulation may cause less harm to tissues and decrease likelihood of seizures. The most popular cranial electrical stimulator is the Fisher Wallace Cranial Stimulator. This device uses sponge electrodes positioned on the scalp and stimulation with square wave frequencies of 15/500/15,000 Hz with a variable maximum output of 40 volts and variable maximum current output of 4 milliamps. Using the Fisher Wallace Cranial Stimulator with the eyes closed patients experience flashing lights. These flashing lights are considered by most investigators to represent phosphenes (seeing lights from cortical stimulation without retinal stimulation), but a component of retinal stimulation cannot be absolutely excluded. (Kanai, Chaieb, Antal, Walsh, & Paulus, 2008; Schutter & Hortensius, 2010)

The pterion and in some instances the adjacent squamous temporal bone is the thinnest area of the cranium and cover the anterior temporal lobe. (Ma, Baillie, & Stringer, 2012) There are anatomic differences in the scalp location of the pterion among races. (Aksu, Akyer, Kale, Geylan, & Gayretli, 2014; Oguz, Sanli, Bozkir, & Soames, 2004; Saxena, Jain, & Chowdhary, 1988; Ukoha et al., 2013) When the scalp over this area is stimulated and the eyes are closed phosphenes occur, and they can be a measurement of cortical stimulation. (Abrahamyan et al., 2011; Antal, Kincses, Nitsche, & Paulus, 2003) In this invention the term “phosphene threshold” has been redefine as the lowest electrical power that elicits phosphenes at a given waveform, frequency and impedance between electrodes. When the sub cortical temporal lobe is stimulated phosphenes, fear, memory recall, complex visual hallucinations and déjà vu are frequently reported. (Gloor, Olivier, Quesney, Andermann, & Horowitz, 1982) Some of these effects may be related to stimulation of the amydala and hippocampus that are located within the temporal lobe rather than stimulation of the temporal lobe cortex. (Lesse, Hoch, Ransohoff, Sloan, & Glussman, 1955)

Bone is a significant insulator such that small changes in the thickness of the skull can have profound changes in the impedance between scalp electrodes. Various conductivity measurements in S/m include, a scalp: skull: brain ratio of 1:1/80: 1 or a skin: muscle: bone: cerebral spinal fluid ratio of 0.1:0.35:0.02:2.0. (Gabriel, Lau, & Gabriel, 1996; Homma et al., 1995)

Optimum preparation and location of scalp electrodes is related to a number of factors including conduction at the skin electrode interface, positioning the electrodes over areas that are associated with lowest impedance between the electrodes and possibly targeting specific brain locations. (Spach, Barr, Haystad, & Long, 1966) Standard electrode pads that are Ag/AgCI ring electrodes can be made more conductive by application of additional silver, a highly conductive metal. Optimum stimulation parameters include waveform, frequency and power that is determined by voltage and current. Whether the stimulation is produce by a medical device or by changes in electromagnetic fields of the environment, scalp stimulation in areas of low impedance that correspond to areas of thinnest cortical bone is likely to be most efficient and require less electrical power to produce brain stimulation.

This invention relates to the optimum preparation and placement of scalp electrodes for cranial electrical stimulation and optimum waveform, frequency, current and voltage for cranial electrical stimulation.

### DRAWINGS

FIG. 1 label 1 shows an area corresponding to the pterion that is the thinnest section of the cranium and in most humans covers the anterior temporal lobe.

### DETAILED DESCRIPTION OF THE INVENTION

Transcranial electrical stimulation requires that scalp electrodes be prepared optimally and placed on the scalp. Optimum preparation and placement decreases the impedance between the electrodes that ensures administration of the lowest electrical power. Minimizing impedance between the electrodes has theoretical advantages because less power is required and injury to brain tissue and likelihood of seizures may be less. The phosphene threshold as define in this invention is one monitor of the effectiveness of cranial electrical stimulation.

When bilateral scalp electrodes were placed in a locations over the pterions it was discovered that the lowest impedance between electrodes was measured. (Table 1) The pterions are the thinness areas of the cranium and this could be the reason for the low impedance over these areas. These low impedance measurements were even more significant because the distance between the electrodes placed over the pterions was much greater than between electrodes placed over the frontal bone. This optimum location of an electrode over the pterion can be identified by the point of intersection of a perpendicular line 40 mm above the midpoint of the zygomatic arch and an oblique line 34 mm from the frontozygomatic suture that is in proximity to the lateral canthus. (Oguz et al., 2004) Impedance between bilateral electrodes can be less than one kiloohm. The location of the pterion varies between races and this need to be considered for optimum electrode placement. (Aksu et al., 2014; Oguz et al., 2004; Ukoha et al., 2013)

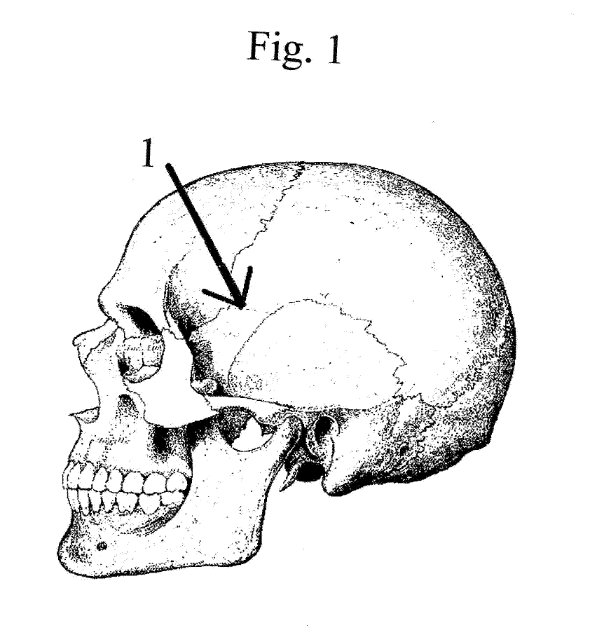

In this invention it was discovered:
1. After cleaning the scalp with a solvent such as isopropyl alcohol and application of conductive liquid silver to standard electrode pads there was a significant decrease impedance between electrodes. (Table 1)
2. Placement of electrode pads over the areas of the scalp which cover the pterions, the thinnest areas of the skull, minimizes the impedance between the electrodes. (Table 1 and FIG. 1)
3. Stimulation of the scalp over the areas covering the pterions with very low electrical power (24-28 mW) was associated with a phosphene threshold. (Table 2)
4. Unlike square wave stimulation, phosphenes were experienced with sine wave and stair wave stimulation only at 15 Hz. (Table 2)
5. The frequencies that produce phosphenes at low electrical power were low frequency between 10-35 Hz and approximate the Schumann resonance frequencies of 7.38 Hz and the associated harmonic frequencies and the frequencies recorded by a standard EEG montage.

### Experimental Data

Average impedance measurements with scalp electrodes (Red Dot™, 3M Company) over pterions and frontal bone are shown in Table 1. Impedance was measured with Commercial Electric MS8260A digital multimeter. Silver paint (18% M.E. Taylor Company) was applied to the center of the scalp electrodes.

TABLE 1
Impedance associated with preparation and placement of scalp electrodes.

| | Pterions | Frontal bone |
|---|---|---|
| Skin preparation | (kiloohms) | (kiloohms) |
| None | 156 | 92 |
| 91% Isopropyl alcohol with | 9 | 19 |
| skin abrasion | | |
| 91% Isopropyl alcohol with | 0.75 | 5 |
| skin abrasion and silver paint | | |

Electric waveforms generated by OWON DDS Arbitrary Waveform Generator Model AG1012A are listed in Table 2. Recorded phosphene thresholds with bilateral electrodes placed over the pterions with 100 mV input and impedance between electrodes of 750 ohms. (Table 2)

TABLE 2
Reported phosphene thresholds at 100 mV with
750 ohm impedance between electrodes.

| | Square wave | | Sine wave | | Stair wave | |
|---|---|---|---|---|---|---|
| | Phos- | Input | Phos- | Input | Phos- | Input |
| Fre- | phene | milliamps | phene | milliamps | phene | milliamps |
| quency | Thresh- | (maxi- | thresh- | (maxi- | thresh- | (maxi- |
| (Hz) | old | mum) | old | mum) | old | mum) |
| 5 | − | 0.49 | − | 0.04 | − | 0.17 |
| 10 | + | 0.28 | − | 0.28 | − | 0.04 |
| 15 | + | 0.30 | + | 0.45 | + | 0.04 |
| 20 | + | 0.27 | − | 050 | − | 0.03 |
| 25 | + | 0.24 | − | 0.53 | − | 0.03 |
| 30 | + | 0.25 | − | 0.54 | − | 0.03 |
| 35 | + | 0.25 | − | 0.55 | − | 0.03 |
| 40 | − | 0.24 | − | 0.55 | − | 0.03 |
| 45 | − | 0.24 | − | 0.55 | − | 0.03 |
| 50 | − | 0.24 | − | 0.55 | − | 0.03 |
| 100 | − | 0.24 | − | 0.55 | − | 0.03 |
| 500 | − | 0.24 | − | 0.54 | − | 0.03 |
| 15,000 | − | 0.23 | − | 0.1 | − | 0.02 |

### Benefits to Society

Cranial electrical stimulation is a proven procedure for the treatment of depression, anxiety, insomnia and chronic pain. Optimum preparation and placement of scalp electrodes that produces the least impedance between electrodes may be safer because the power required to perceive phosphenes is less. The square wave frequencies that produce a cortical response measured by the phosphene threshold at 100 mV and between 0.28-.25 ma (25-28 mW) were between 10-35 Hz. These frequencies are within the range of frequencies found in the Schumann resonance and its harmonics that are commonly measured at 7.83, 14, 21, 26, 33, and 39 Hz. From an evolutionary standpoint external electromagnetic influences that may be beneficial to the survival of an organism might selectively stimulate thin areas of the cranium such as the pterions. In most humans, this area overlies the anterior temporal lobe near structures of the limbic system such as the amygdala and hippocampus. The predominant experience associated with stimulation of these structures is fear that is crucial for survival. In addition to the frequencies associated with phosphenes and Schumann frequencies, the frequencies that are commonly recorded on EEG are Delta: 1-3 Hz, Theta: 4-7 Hz, Alpha1: 8-9 Hz, Alpha2: 10-12 Hz, Beta1: 13-17 Hz, Beta2: 18-30 Hz, Gamma1: 31-40 Hz, and Gamma2: 41-50 Hz suggesting that wave interference may be possible from sources external to the brain.

### REFERENCES

Abrahamyan, A., Clifford, C. W., Ruzzoli, M., Phillips, D., Arabzadeh, E., & Harris, J. A. (2011). Accurate and rapid estimation of phosphene thresholds (REPT). PLoS One, 6(7), e22342. doi: 10.1371/journal.pone.0022342
Aksu, F., Akyer, S. P., Kale, A., Geylan, S., & Gayretli, O. (2014). The localization and morphology of pterion in adult West Anatolian skulls. J Craniofac Surg, 25(4), 1488-1491. doi: 10.1097/SCS.0000000000000790
Antal, A., Kincses, T. Z., Nitsche, M. A., & Paulus, W. (2003). Manipulation of phosphene thresholds by transcranial direct current stimulation in man. Exp Brain Res, 150(3), 375-378. doi: 10.1007/s00221-003-1459-8
DaSilva, A. F., Volz, M. S., Bikson, M., & Fregni, F. (2011). Electrode positioning and montage in transcranial direct current stimulation. J Vis Exp (51). doi: 10.3791/2744
Gabriel, S., Lau, R. W., & Gabriel, C. (1996). The dielectric properties of biological tissues: II. Measurements in the frequency range 10 Hz to 20 GHz. Phys Med Biol, 41(11), 2251-2269.
Gloor, P., Olivier, A., Quesney, L. F., Andermann, F., & Horowitz, S. (1982). The role of the limbic system in experiential phenomena of temporal lobe epilepsy. Ann Neurol, 12(2), 129-144. doi: 10.1002/ana.410120203
Homma, S., Musha, T., Nakajima, Y., Okamoto, Y., Blom, S., Flink, R., & Hagbarth, K. E. (1995). Conductivity ratios of the scalp-skull-brain head model in estimating equivalent dipole sources in human brain. Neurosci Res, 22(1), 51-55.
Kanai, R., Chaieb, L., Antal, A., Walsh, V., & Paulus, W. (2008). Frequency-dependent electrical stimulation of the visual cortex. Curr Blot 18(23), 1839-1843. doi: 10.1016/j.cub.2008.10.027
Lesse, S., Hoch, P., Ransohoff, J., Sloan, N., & Glussman, M. (1955). Observations of temporal lobe stimulation and psychosurgery under local anesthesia. Trans Am Neurol Assoc(80th Meeting), 48-50.
Ma, S., Baillie, L. J., & Stringer, M. D. (2012). Reappraising the surface anatomy of the pterion and its relationship to the middle meningeal artery. Clin Anat, 25(3), 330-339. doi: 10.1002/ca.21232
Oguz, O., Sanli, S. G., Bozkir, M. G., & Soames, R. W. (2004). The pterion in Turkish male skulls. Surg Radiol Anat, 26(3), 220-224. doi: 10.1007/s00276-003-0210-2
Saxena, S. K., Jain, S. P., & Chowdhary, D. S. (1988). A comparative study of pterion formation and its variations in the skulls of Nigerians and Indians. Anthropol Anz, 46(1), 75-82.
Schutter, D. J., & Hortensius, R. (2010). Retinal origin of phosphenes to transcranial alternating current stimulation. Clin Neurophysiol, 121(7), 1080-1084. doi: 10.1016/j.clinph.2009.10.038
Spach, M. S., Barr, R. C., Haystad, J. W., & Long, E. C. (1966). Skin-electrode impedance and its effect on recording cardiac potentials. Circulation, 34(4), 649-656.
Ukoha, U., Oranusi, C. K., Okafor, J. I., Udemezue, O. O., Anyabolu, A. E., & Nwamarachi, T. C. (2013). Anatomic study of the pterion in Nigerian dry human skulls. Niger J Clin Pract, 16(3), 325-328. doi: 10.4103/1119-3077.113455

Having described my invention, I claim:

1. A method to prepare and locate optimum placement of scalp electrodes measured by the lowest impedance between the electrodes for purposes of transcranial electrical stimulation.

2. The method of claim 1 that uses conductive silver paint applied to standard electrode pads and placement of electrode pads on the scalp over the pterions.
